(19) United States
(12) Patent Application Publication
(10) Pub. No.: US 2023/0017666 A1
(43) Pub. Date: Jan. 19, 2023

(54) AN UBIQUITIN LIGASE INHIBITOR FOR USE FOR PREVENTING AND/OR TREATING A DISEASE LINKED WITH CEREBRAL HYPOPERFUSION

(71) Applicants: CENTRE NATIONAL DE LA RECHERCHE SCIENTIFIQUE, Paris (FR); INSTITUT NATIONAL DE LA SANTE ET DE LA RECHERCHE MEDICALE (INSERM), Paris (FR); COMMISSARIAT À L'ÉNERGIE ATOMIQUE ET AUX ÉNERGIES ALTERNATIVES, Paris (FR); UNIVERSITÉ DE BORDEAUX, Bordeaux (FR); CHU DE BORDEAUX, Talance Cedex (FR); INRIA INSTITUT NATIONAL DE RECHERCHE EN INFORMATIQUE ET EN AUTOMATIQUE, Le Chesany (FR)

(72) Inventors: Bruno BONTEMPI, Bordeaux (FR); Cécile DUPLAA, Pessac (FR); Thierry COUFFINHAL, Pessac (FR); Jean-Luc MOREL, Bordeaux (FR)

(21) Appl. No.: 17/786,796

(22) PCT Filed: Dec. 18, 2020
(86) PCT No.: PCT/EP2020/086994

§ 371 Date: Jun. 17, 2022

(30) Foreign Application Priority Data
Dec. 18, 2019 (EP) 19306684.2

(51) Int. Cl. C12N 15/113; A61P 25/28; C12N 15/115

(52) U.S. Cl. CPC C12N 15/1137; A61P 25/28; C12N 15/115; C12N 2310/14; C12N 2310/531; C12N 2310/141; C12N 2310/11

(57) ABSTRACT

The present invention concerns an ubiquitin ligase inhibitor for use for preventing and/or treating a disease linked with cerebral hypoperfusion, and an in vitro screening method for the identification of a candidate compound suitable for preventing and/or treating a disease linked with cerebral hypoperfusion.

The present invention concerns an ubiquitin ligase inhibitor for use for preventing and/or treating a disease linked with cerebral hypoperfusion, and an in vitro screening method for the identification of a candidate compound suitable for preventing and/or treating a disease linked with cerebral hypoperfusion.

Hypoperfusion is the state of insufficient blood flow to the tissues of the body as a result of problems with the circulatory system. It can be divided into four main types based on the underlying cause: low volume, cardiogenic, obstructive, and distributive. In severe cases it can induce a state of shock, tissue necrosis, dementia (when it concerns the brain), or even death. Hypoperfusion most often designates cerebral hypoperfusion and in particular, cerebral ischemia, the decline in vascularity of a region of the brain (which seems to be a long-term factor of dementia and Alzheimer's disease). Hypoperfusion can be transient (due to compression) or be more durable, and then threaten an organ and its functions, hypoperfusion can even be life-threatening.

Alzheimer's disease (AD) is one of the common causes of age-related cognitive impairment and has been considered a neurodegenerative disease affecting neurons. However, vascular dysfunction and damage are frequently associated with AD; individuals with AD display cerebral blood flow reductions, loss of vascular integrity and blood brain barrier (BBB) disruption. Cerebral vascular changes in AD have then been mostly attributed to the vasculotoxic effects of amyloïd β (Aβ) or Tau deposition. But, epidemiological study show that vascular risk factors such as midlife hypertension, obesity, diabete mellitus are each associated with a 2 to 3 fold increased risk of elevated cerebral amyloid later in life. Brain imaging studies have identified vascular dysregulation as an early pathological event in presymptomatic individuals at risk for AD. Experimental data also report that transgenic mice have an alteration of neurovascular regulation before the expression of cognitive deficits and amyloid plaques. Collectively, these observations suggest the possibility that BBB dysfunction may be an early pre symptomatic correlate of AD progression or an aggravating pathogenic factor in AD. Experimental studies still need to be conducted to establish a causal relationship between BBB dysfunction and AD pathology and to gain insight into the underlying biological mechanisms that affect BBB stability.

In the central nervous system, the BBB is characterized by specialized endothelial cells with continuous intercellular tight junctions, lack of fenestration and low rates of transcytosis, which greatly limits both the paracellular and transcellular movement of molecules through the endothelial cell layer. Maintaining BBB integrity is crucial for selective metabolic control of the brain interstitial fluid composition, required for proper synaptic functioning and neuronal connectivity. When these junctions are disrupted, the barrier function is compromised and edema occurs which can initiate pathways of neurodegeneration. Thus, great interest arises to delineate the signaling pathways regulating physiological function of the BBB and gain insights into pathological conditions causing loss or breakdown of the BBB. Wnt/B-catenin signaling is involved in BBB induction, maturation and maintenance that is activated by ligands Wnt7a, Wnt7b, Norrin. Transcriptomic analyses assess that the most prominent up-regulated pathways in brain endothelial cells are the “Wnt signaling” and “adherens junction” pathways. Recently it was reported that the BBB phenotype state of endothelial cells is under the control of, at least, the Wnt/β-catenin (canonical Wnt) signalling.

The E3 ubiquitin ligase called PDZRN3 was reported as a core mediator of the non-canonical Wnt pathway in endothelial cells which represses canonical Wnt signaling. This ubiquitin ligase acts downstream the PAR3 polarity complex and has been implicated in vascular permeability.

The inventors clarified cerebral vessel mechanisms by which cerebral hypoperfusion (HP) may cause cognitive impairment. Surprisingly, they demonstrated that targeting PDZRN3 selectively in vascular cells may limit loss of barrier function and tissue damage induced under cerebral HP, and can prevent neurological deficits induced in AD context.

According to one aspect, the invention thus concerns PDZRN3 inhibitor for use for preventing and/or treating a disease linked with cerebral hypoperfusion.

According to another aspect, the invention concerns a pharmaceutical composition comprising a PDZRN3 inhibitor and a pharmaceutically acceptable carrier.

The invention concerns the use of a PDZRN3 inhibitor for the manufacture of a medicament for preventing and/or treating a disease linked with cerebral hypoperfusion.

The invention also concerns a method for preventing and/or treating a disease linked with cerebral hypoperfusion in a subject comprising the administration of PDZRN3 inhibitor. Preferably, the method for preventing and/or treating a disease linked with cerebral hypoperfusion comprises the administration of a therapeutically effective amount of a PDZRN3 inhibitor to a subject in need thereof.

The invention also concerns an in vitro screening method for the identification of a candidate compound suitable for preventing and/or treating disease linked with cerebral hypoperfusion, said method comprising:
a. culturing endothelial cells, in the presence and in the absence of a candidate compound;
b. measuring the level of expression of Pdznr3 or PDZRN3 biological activity in endothelial cells cultured in the presence and in the absence of the candidate compound;
c. comparing the level of expression of Pdznr3 in endothelial cells in the presence the candidate compound, with the level of expression of Pdznr3 endothelial cells in the absence of the candidate compound;
or comparing the level of PDZRN3 biological activity in endothelial cells in the presence the candidate compound, with the level of PDZRN3 biological activity in endothelial cells in the absence of the candidate compound; and
d. identifying the candidate compound as suitable for preventing and/or treating disease linked with cerebral hypoperfusion if the level of expression of Pdznr3 in endothelial cells in the presence the candidate compound is decreased compared with the level of expression of Pdznr3 in endothelial cells in the absence of the candidate; or identifying the candidate compound as suitable for preventing and/or treating disease linked with cerebral hypoperfusion if the level of PDZRN3 biological activity in endothelial cells in the presence the candidate compound is decreased compared with the level PDZRN3 biological activity in endothelial cells in the absence of the candidate.

### DETAILED DESCRIPTION OF THE INVENTION

The invention concerns a PDZRN3 inhibitor for use for preventing and/or treating a disease linked with cerebral hypoperfusion.

By “PDZRN3” is meant herein any naturally occurring isoform of the E3 ubiquitin ligase protein PDZRN3, allelic variants thereof, splice variants thereof and orthologous proteins. Typically, the sequence of the human E3 ubiquitin ligase PDZRN3 is as set forth under Genbank Accession Number NM_015009.3 as of 22 Aug. 2019.

A “PDZRN3 inhibitor” refers to any compound that has a biological effect to inhibit the expression of a PDZRN3 gene (Pdzrn3) and/or inhibit or reduces a PDZRN3 biological activity. In one embodiment of the invention, said inhibitor of Pdzrn3 expression is a Small inhibitory RNA (siRNA), a small hairpin RNA (shRNA), a micro RNA (mi RNA), an antisense oligonucleotide or an aptamer. In one embodiment, said inhibitor has a nucleotide sequence having at least 80% and preferably at least 95% of complementary residues with Pdzrn3's messenger RNA or part thereof.

Nucleic acid sequence identity can be calculated by methods well-known to one of skill in the art. The percentage of identity may be calculated by performing a pairwise global alignment based on the Needleman-Wunsch alignment algorithm to find the optimum alignment (including gaps) of two sequences along their entire length, for instance using Needle, and using the DNAFULL matrix with a gap opening penalty of 10 and a gap extension penalty of 0.5.

Small inhibitory RNAs (siRNAs) can function as inhibitors of gene expression for use in the invention. Gene expression can be reduced with a small double stranded RNA (dsRNA), or a vector or construct causing the production of a small double stranded RNA, such that gene expression is specifically inhibited (i.e. RNA interference or RNAi). Methods for selecting an appropriate dsRNA or dsRNA-encoding vector are well known in the art for genes whose sequence is known.

Inhibitors of Pdzrn3 for use in the invention may be based on antisense oligonucleotide (ODNs) constructs. Antisense oligonucleotides, including antisense RNA molecules and antisense DNA molecules, would act to directly block the activity of Pdzrn3 by binding to Pdzrn3 mRNA and thus preventing binding leading and to mRNA degradation. For example, antisense oligonucleotides of at least about 15 bases and complementary to unique regions of the Pdzrn3 transcript sequence can be synthesized, e.g., by conventional phosphodiester techniques and administered by e.g., intravenous injection or infusion. Methods for using antisense techniques for specifically inhibiting gene expression of genes whose sequence is known are well known in the art. It should be further noted that antisense oligonucleotides may be modified with phosphorothioate to prevent their in vivo hydrolysis by nucleases. Such modifications are well known in the art. Antisense oligonucleotides useful as inhibitors of Pdzrn3 can be prepared by known methods. These include techniques for chemical synthesis such as, e.g., by solid phase phosphoramadite chemical synthesis. They can also be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters.

In one embodiment, said PDZRN3 inhibitor inhibits PDZRN3 biological activity, or inhibits PDZRN3 interaction with its targets. In particular in this embodiment, said PDZRN3 inhibitor is a chemical molecule, a peptide, a protein, an aptamer, an antibody or an antibody fragment. As used herein, the terms “inhibition of the biological activity” means preventing or reducing one or several biological effects of PDZRN3. As used herein, the terms “inhibition of the interaction” means preventing or reducing the direct or indirect association of one or more molecules, nucleic acids, peptides or proteins. This inhibition can be realized by competition or by fixing to one of the molecules. In a preferred embodiment, the inhibitor is a peptide. In a particular embodiment, the inhibitor is an antibody or antibody fragment.

By “peptide”, it is meant an amino acid sequence comprising from 2 to 30 amino acids. By “protein”, it is meant an amino acid sequence comprising at least 31 amino acids, preferably 50 to 500 amino acids.

By “antibody” it is meant immunoglobulin molecules and immunologically active portions of immunoglobulin molecules, i.e., molecules that contain an antigen binding site which immunospecifically binds an antigen. As such, the term antibody encompasses not only whole antibody molecules, but also antibody fragments as well as variants (including derivatives) of antibodies and antibody fragments. In particular, the antibody according to the invention may correspond to a polyclonal antibody, a monoclonal antibody (e.g. a chimeric, humanized or human antibody), a fragment of a polyclonal or monoclonal antibody or a diabody.

By “antibody fragments” it is meant a portion of an intact antibody, preferably the antigen binding or variable region of the intact antibody. Examples of antibody fragments include Fv, Fab, F(ab)₂, Fab′, Fd, dAb, dsFv, scFv, sc(Fv)₂, CDRs, diabodies and multi-specific antibodies formed from antibodies fragments.

By “aptamers” it is meant the class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., Science, 1990, 249(4968):505-10. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., Clin. Chem., 1999, 45(9):1628-50. Then, after identifying the aptamers directed against HIRA as described above, those skilled in the art can readily select the ones inhibiting HIRA. Preferably, the aptamer is an oligonucleotide or polypeptide from 10 to 30 kDa.

Methods for determining whether a compound is a PDZRN3 inhibitor are well known by the person skilled in the art. For example, the person skilled in the art can assess whether a compound decreases Pdzrn3 expression. According to the invention, the “level of expression of Pdzrn3” is determined by quantifying Pdzrn3 mRNA, or a fragment thereof, or by quantifying the amount of PDZRN3. For example, the amount of Pdzrn3 mRNA can be quantified by RT-qPCR. Northern Blot, Western Blot of PDZRN3 and/or enzyme-linked immunosorbent assay (ELISA) can also be used.

Other methods for determining whether a compound is a PDZRN3 inhibitor may be for example, by measuring the biological activity of PDZRN3, through measuring one of the phenomenon in which PDZRN3 is known to play a role, and can comprises any method well-known to one of skill in the art. For instance, the inventors have demonstrated that PDZRN3 is implicated in the maintain of tight junction protein complex in endothelial cell contacts. The biological activity of PDZRN3 may be assessed through measuring endothelial cell permeability or through the induction of c-jun or induction of c-jun reporter activity in vitro.

In the context of the invention, the PDZRN3 inhibitor reduces brain inflammation. In particular, it reduces the number of brain lesions, reduces neuronal loss and/or reduces the number of reactive astrocytes.

In a particular embodiment, the inhibitor is optimized to facilitate its production or its action, or to slow it degradation in the subject. For example, the inhibitor can be linked or fused to a tag allowing the targeting of particular region or cells of the subject. In particular, the tag can allow the targeting of endothelial cells, or cells of the brain, or cerebral endothelial cells, or cells of cortical regions, or cells of hippocampal regions and in particular of the CA1 hippocampal region.

In the context of the invention, the term “treating” or “treatment”, refers to a therapeutic use (i.e. on a subject having a given disease) and means reversing, alleviating, inhibiting the progress of one or more symptoms of such disorder or condition. Therefore, treatment does not only refer to a treatment that leads to a complete cure of the disease, but also to treatments that slow down the progression of the disease and/or prolong the survival of the subject.

By “preventing” is meant a prophylactic use (i.e. on a subject susceptible of developing a given disease).

The term “a disease linked with cerebral hypoperfusion” refers to any diseases or injuries due to insufficient blood flow to the brain or part of the brain. In particular, these diseases comprise ischemic strokes, hemorrhagic strokes, cranial trauma, vascular dementia, multiple sclerosis, Parkinson's disease and Alzheimer's disease. In an embodiment, the disease is selected among ischemic strokes, hemorrhagic strokes and cranial trauma. In a preferred embodiment, the disease is selected among vascular dementia, multiple sclerosis, Parkinson's disease and Alzheimer's disease. In another embodiment, the disease is vascular dementia or Alzheimer's disease. In a particular embodiment, the disease is Alzheimer's disease.

Preferably, the PDZRN3 inhibitor for use for preventing and/or treating a disease linked with cerebral hypoperfusion is used on a subject. The “subject” or “individual” may be, for example, a human or non human mammal, such as a rodent (mouse, rat), a feline, a canine or a primate, affected by or likely to be affected by a cerebral hypoperfusion. Typically, the subject is a human. In a particular embodiment the subject is a human of at least 40 years old, or of at least 50 years old, or of at least 60 years old, or of at least 70 years old.

The PDZRN3 inhibitor is advantageously formulated in a pharmaceutical composition, preferably together with a pharmaceutically acceptable carrier.

Thus, the invention also concerns a pharmaceutical composition comprising a PDZRN3 inhibitor and a pharmaceutically acceptable carrier. In a particular embodiment, the pharmaceutical composition also comprises other drugs for the treatment of vascular dementia, Parkinson's disease, Alzheimer's disease or Multiple sclerosis.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier, excipient or diluent refers to a non-toxic solid, semi-solid or liquid filler, diluent, encapsulating material or formulation auxiliary of any type.

Pharmaceutically acceptable carriers and excipient that may be used in the compositions of this invention include, but are not limited to, ion exchangers, alumina, aluminium stearate, lecithin, self-emulsifying drug delivery systems (SEDDS) such as d-a-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

As appreciated by skilled artisans, the PDZRN3 inhibitor or the pharmaceutical composition is suitably formulated to be compatible with the intended route of administration. Examples of suitable routes of administration include oral route, intranasal route, intraocular route, parenteral route, and including intramuscular, subcutaneous, intravenous, intraperitoneal or local injections. In a particular embodiment, the PDZRN3 inhibitor or the pharmaceutical composition is suitably formulated to be compatible with the oral route or local injections. The oral route can be used, provided that the composition is in a form suitable for oral administration, i.e. able to protect the active principle from the gastric and intestinal enzymes. Preferably, the PDZRN3 inhibitor for the use according to the invention is administered by topical route, oral route, intranasal route, intraocular route, parenteral route, or by intramuscular, subcutaneous, intravenous, intraperitoneal or local injections. More preferably, the PDZRN3 inhibitor for the use according to the invention is administered by oral route or local injections.

Preferably, the pharmaceutical composition contains carriers that are pharmaceutically acceptable for an injectable formulation. They may in particular be sterile, isotonic, saline solutions (monosodium phosphate, disodium phosphate, sodium chloride, potassium chloride, calcium chloride or magnesium chloride etc., or mixtures of such salts), or dry, in particular lyophilized, compositions which by means of the addition, as appropriate, of sterilized water or physiological saline, can form injectable solutes.

The doses used for the administration can be adapted as a function of various parameters, and in particular as a function of the mode of administration used, of the relevant pathology, or alternatively of the desired duration of treatment. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

It is routine for those skilled in the art to adjust the nature and amount of excipients in the pharmaceutical composition so as not to affect the desired properties thereof, notably with regard to the stability of the PDZRN3 inhibitor according to the invention, and the route of administration considered.

The invention concerns the use of a PDZRN3 inhibitor for the manufacture of a medicament for preventing and/or treating a disease linked with cerebral hypoperfusion.

The invention also concerns a method for preventing and/or treating a disease linked with cerebral hypoperfusion in a subject comprising the administration of a PDZRN3 inhibitor. Preferably, the method for preventing and/or treating a disease linked with cerebral hypoperfusion comprise the administration of a therapeutically effective amount of a PDZRN3 inhibitor to a subject in need thereof.

Preferably, an effective amount, preferably a therapeutically effective amount of the PDZRN3 inhibitor of the invention is administered. An “effective amount” refers to an amount effective, at dosages and for periods of time necessary, to achieve the desired therapeutic or prophylactic result.

A “therapeutically effective amount” of a PDZRN3 inhibitor of the invention may vary according to factors such as the disease state, age, sex, and weight of the individual, and the ability of the PDZRN3 inhibitor, to elicit a desired therapeutic result. A therapeutically effective amount encompasses an amount in which any toxic or detrimental effects of the PDZRN3 inhibitor are outweighed by the therapeutically beneficial effects. A therapeutically effective amount also encompasses an amount sufficient to confer benefit, e.g., clinical benefit.

The invention also concerns an in vitro screening method for the identification of a candidate compound suitable for preventing and/or treating disease linked with cerebral hypoperfusion, said method comprising:
a. culturing endothelial cells, in the presence and in the absence of a candidate compound;
b. measuring the level of expression of Pdznr3 or PDZRN3 biological activity in endothelial cells cultured in the presence and in the absence of the candidate compound;
c. comparing the level of expression of Pdznr3 in endothelial cells in the presence the candidate compound, with the level of expression of Pdznr3 endothelial cells in the absence of the candidate compound;
or comparing the level of PDZRN3 biological activity in endothelial cells in the presence the candidate compound, with the level of PDZRN3 biological activity in endothelial cells in the absence of the candidate compound; and
d. identifying the candidate compound as suitable for preventing and/or treating disease linked with cerebral hypoperfusion if the level of expression of Pdznr3 in endothelial cells in the presence the candidate compound is decreased compared with the level of expression of Pdznr3 in endothelial cells in the absence of the candidate; or identifying the candidate compound as suitable for preventing and/or treating disease linked with cerebral hypoperfusion if the level of PDZRN3 biological activity in endothelial cells in the presence the candidate compound is decreased compared with the level PDZRN3 biological activity in endothelial cells in the absence of the candidate.

The invention concerns an in vitro screening method for the identification of a candidate compound suitable for preventing and/or treating disease linked with cerebral hypoperfusion, said method comprising:
a. culturing endothelial cells, in the presence and in the absence of a candidate compound;
b. measuring the level of expression of Pdznr3 in endothelial cells cultured in the presence and in the absence of the candidate compound;
c. comparing the level of expression of Pdznr3 in endothelial cells in the presence the candidate compound, with the level of expression of Pdznr3 endothelial cells in the absence of the candidate compound; and
d. identifying the candidate compound as suitable for preventing and/or treating disease linked with cerebral hypoperfusion if the level of expression of Pdznr3 in endothelial cells in the presence the candidate compound is decreased compared with the level of expression of Pdznr3 in endothelial cells in the absence of the candidate.

The invention also concerns an in vitro screening method for the identification of a candidate compound suitable for preventing and/or treating disease linked with cerebral hypoperfusion, said method comprising:
a. culturing endothelial cells, in the presence and in the absence of a candidate compound;
b. measuring the level of PDZRN3 biological activity in endothelial cells cultured in the presence and in the absence of the candidate compound;
c. comparing the level of PDZRN3 biological activity in endothelial cells in the presence the candidate compound, with the level of PDZRN3 biological activity in endothelial cells in the absence of the candidate compound; and
d. identifying the candidate compound as suitable for preventing and/or treating disease linked with cerebral hypoperfusion if the level of PDZRN3 biological activity in endothelial cells in the presence the candidate compound is decreased compared with the level PDZRN3 biological activity in endothelial cells in the absence of the candidate.

Throughout the instant application, the term “comprising” is to be interpreted as encompassing all specifically mentioned features as well optional, additional, unspecified ones. As used herein, the use of the term “comprising” also discloses the embodiment wherein no features other than the specifically mentioned features are present (i.e. “consisting of”).

Furthermore the indefinite article “a” or “an” does not exclude a plurality.

The invention will be further illustrated in view of the following figures and examples.

### FIGURES

FIG. 1.

Experimental scheme for tamoxifen administration to postnatal Pdzrn3 iECKO and IECOE mice; timeline for evaluation of spatial recognition memory in the Y-maze paradigm before and after implantation of ameroid constrictors (BCAS) is shown.

FIG. 2.

Spatial recognition memory performance in the Y-maze exploration test;

left side: deletion of Pdzrn3 (iECKO) protects against chronic hypoperfusion-induced loss of spatial novelty preference in Y-maze evaluated 10 days after BCAS.

right side: Ectopic expression of Pdzrn3 (iECOE) worsened recognition memory performance evaluated 7 days after BCAS.

Results are presented as a recognition index calculated as follows: (time in novel arm−time in familiar arms)/(time in novel arm+time in familiar arms). Student's t-test was performed. Each group contained 9-16 mice and each mouse is indicated as a dot in the graphs.

FIG. 3.

NeuN+ cells were quantified as a percentage of the area occupied in the hippocampal CA1 area before (sham) and after BCAS from iECKO vs their respective littermate mice (left side) and from iECOE vs their respective control mice (right side). Data shown in the graphs are obtained in sham condition, from 3 mice per group; in BCAS condition, from n=13 IECKO vs n=15 littermate mice and from n=6 ICOE vs n=5 control mice.

FIG. 4.

GFAP were quantified in hippocampal area after BCAS in iECKO (left side) and iECOE (right side) mice versus their respective littermates. The size of each group is the same as indicated in FIG. 3.

FIG. 5.

Fibrinogen release was quantified as a percentage of the area occupied in hippocampal region from Pdzrn3 iECKO (n=4) versus littermates (n=7) mice at 21 days after BCAS. Unpaired t-test was performed. *P 0.05. Data are presented as mean±s.e.m (Student's t-test).

FIG. 6.

Fibrinogen was quantified as a percentage of the area occupied either in cortical region (left side) or in hippocampal region (right side) before (sham) and after BCAS.

Quantification was done in brains from non operated sham control (n=3) vs iECOE (n=3) mice and in brains recovered after 14 days of BCAS from control (n=7) vs iECOE (n=6) mice. Data for two brain slices per mouse were recorded and averaged to produce a single value for each mouse. One-way ANOVA was performed. Data are presented as mean±s.e.m.

FIG. 7.

IgG was quantified as a percentage of the area occupied either in cortical region (left side) or in hippocampal region (right side) before (sham) and after BCAS.

Quantification was done in brains from non operated sham control (n=3) vs iECOE (n=3) mice and in brains recovered after 14 days of BCAS from control (n=7) vs iECOE (n=6) mice. Data for two brain slices per mouse were recorded and averaged to produce a single value for each mouse. One-way ANOVA was performed. Data are presented as mean±s.e.m.

FIG. 8.

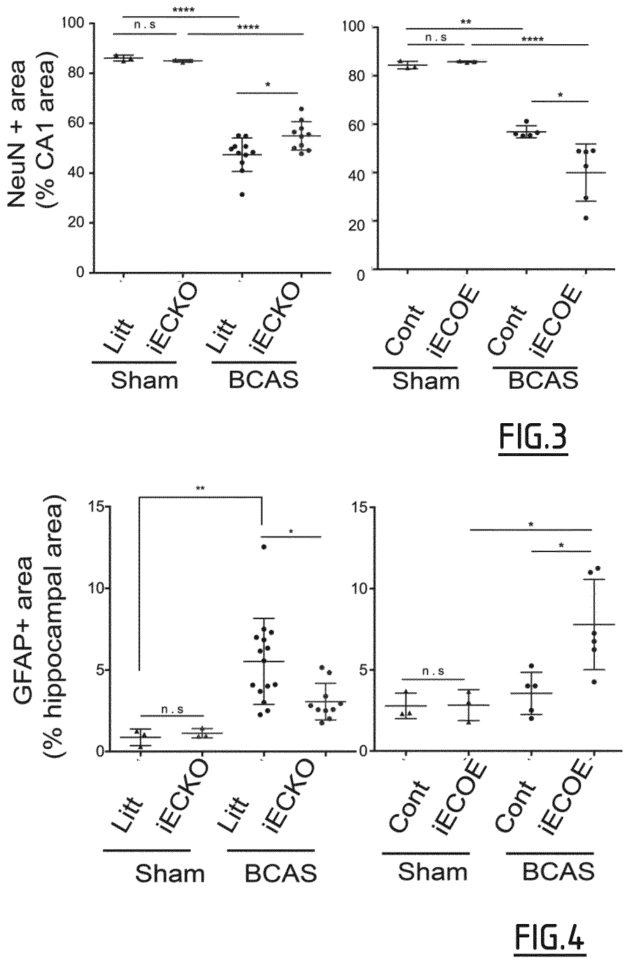

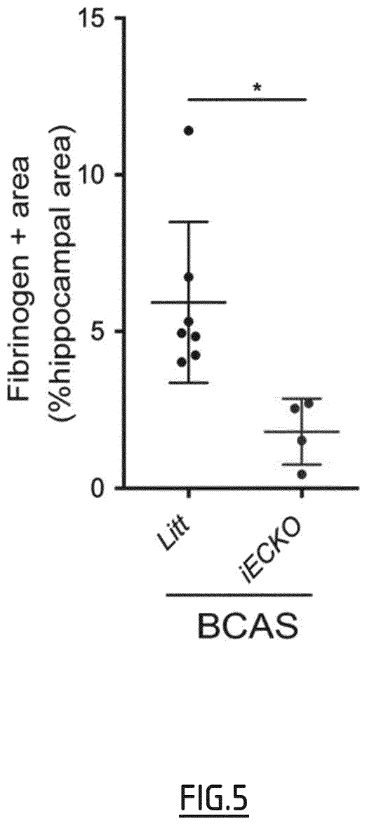

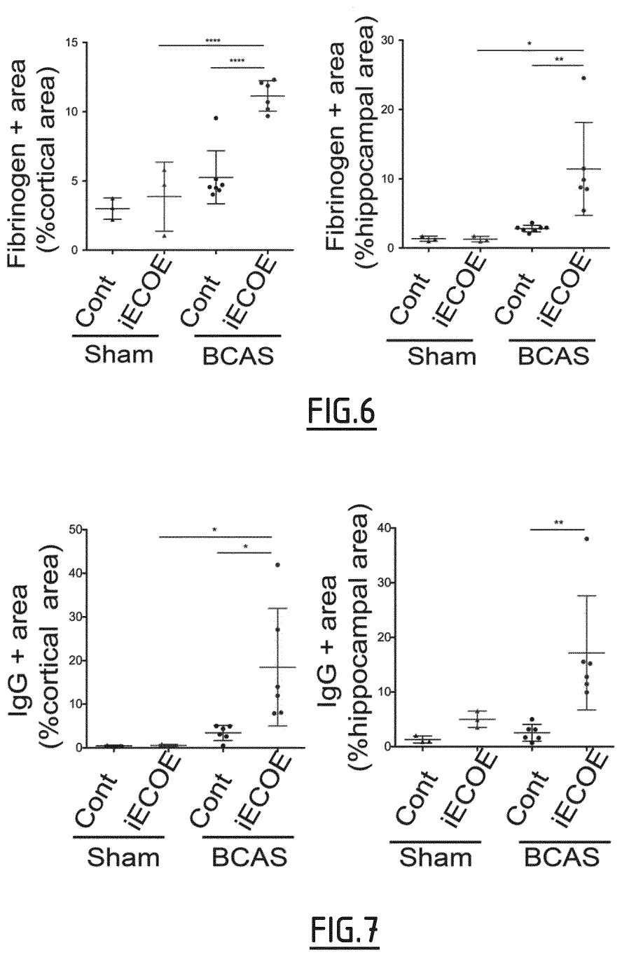

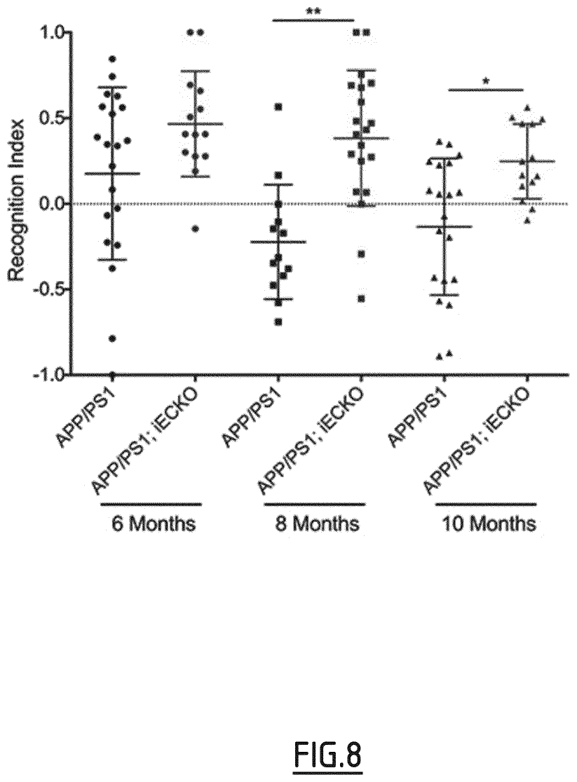

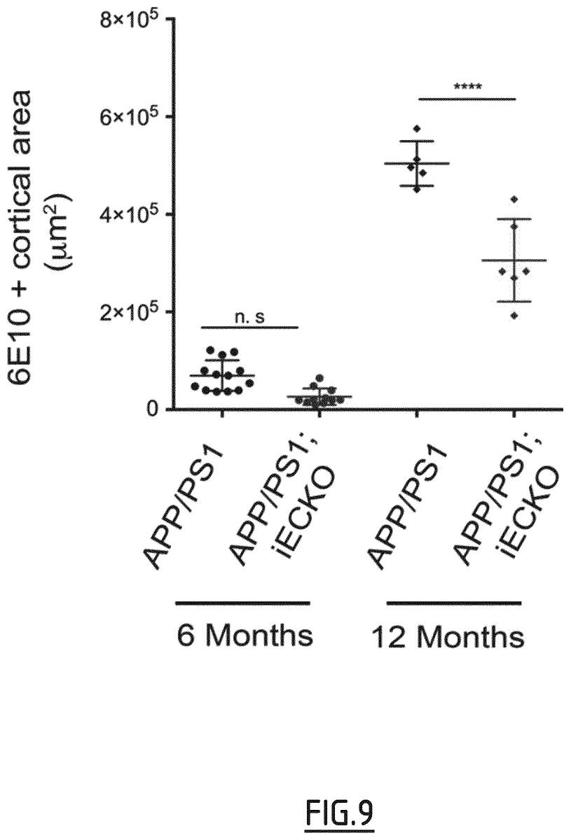

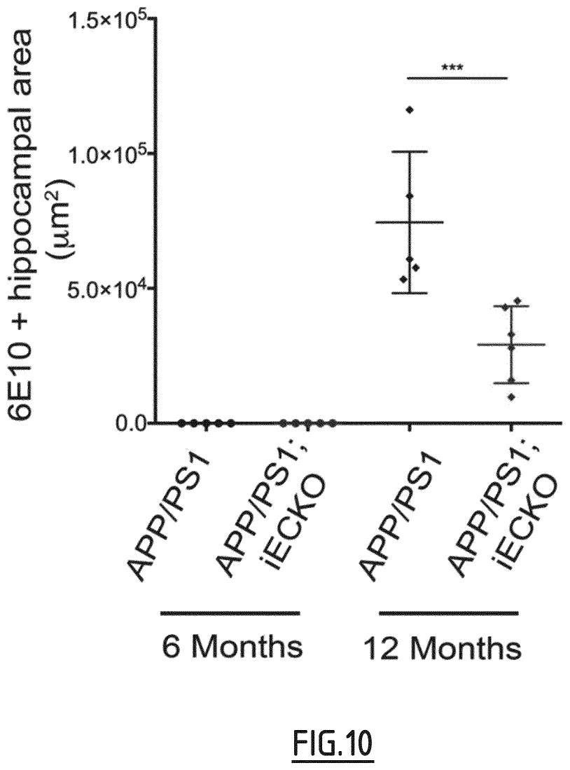

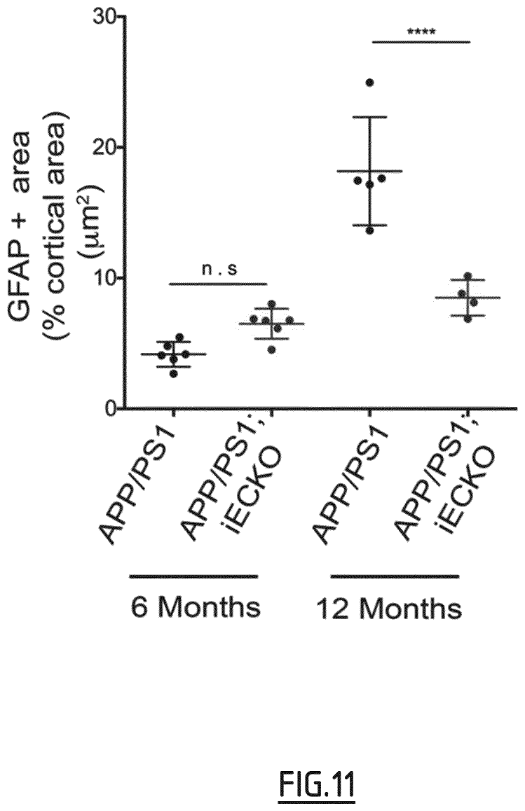

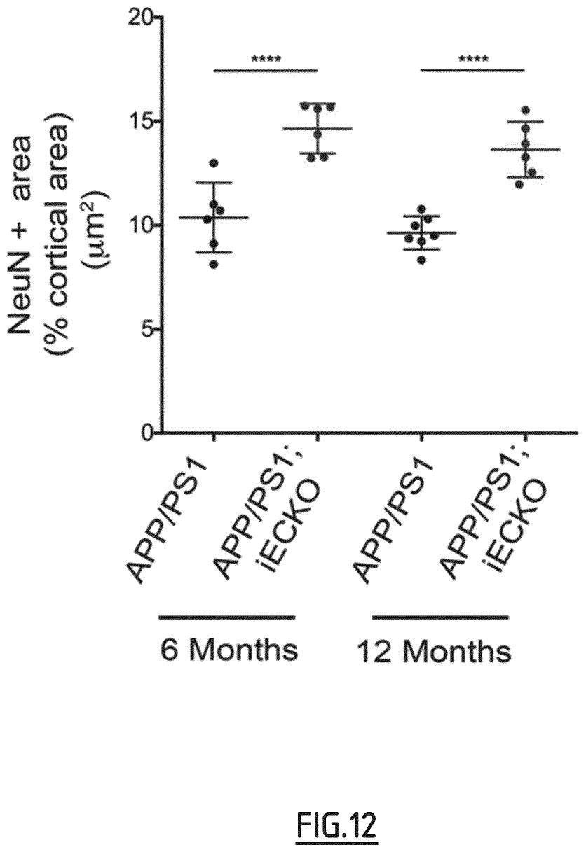

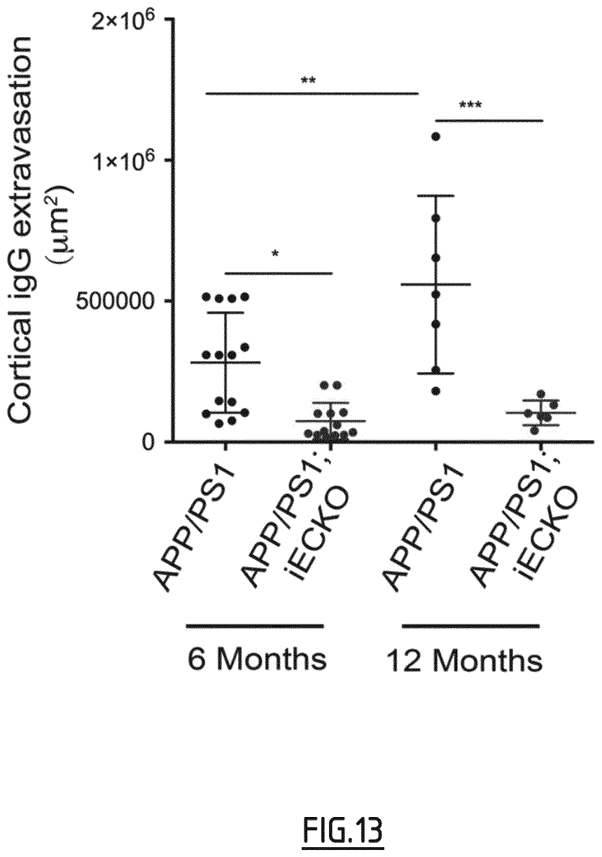

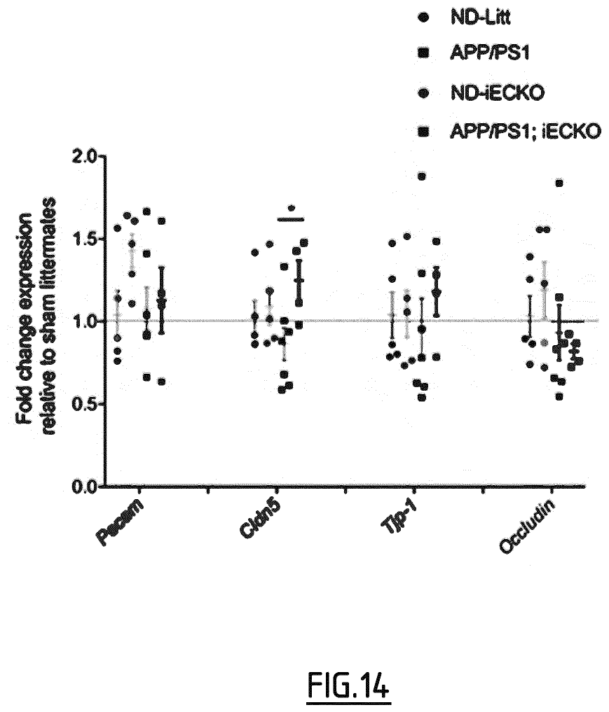

Y-maze exploration test: deletion of Pdzrn3 (iECKO) protects against AD-induced loss of spatial novelty preference in Y-maze evaluated in APP/PS1; IECKO vs APP/PS1 groups at 6, 8 and 10 months of age. Each point represents a different mouse. Data are presented as mean±s.e.m. Recognition index calculated as described in FIG. 2. Two-way ANOVA was performed.

FIG. 9.

Surface of amyloid plaque staining was quantified in entire cortex with the anti-amyloid monoclonal 6E10 antibody. One-way ANOVA was performed. Data are presented as mean±s.e.m.

FIG. 10.

Surface of amyloid plaque staining was quantified in hippocampal region with the anti-amyloid monoclonal 6E10 antibody. One-way ANOVA was performed. Data are presented as mean±s.e.m.

FIG. 11.

GFAP positive staining was quantified as a percentage of the area occupied in cortical region in APP/PS1; iECKO vs their age-matched littermates APP/PS1 at 6 and 12 months. Each point represents a different mouse. Data are presented as mean±s.e.m. Two-way ANOVA was performed.

FIG. 12.

NeuN positive staining was quantified as a percentage of the area occupied in cortical region in APP/PS1; iECKO vs their age-matched littermates APP/PS1 at 6 and 12 months. Each point represents a different mouse. Data are presented as mean±s.e.m. Two-way ANOVA was performed.

FIG. 13.

IgG extravasion was quantified as a percentage of the area occupied in cortical region in APP/PS1; iECKO vs their age-matched littermates APP/PS1 at 6 and 12 months. Each point represents a different mouse. Data are presented as mean±s.e.m. Two-way ANOVA was performed.

FIG. 14.

Transcript levels in brain capillaries isolated from either non-operated ND-iECKO (n=5) vs ND-littermates (n=5) and APP/PS1; iECKO (n=4) vs APP/PS1 (n=5) mice. Data are normalized to that of PO and presented as the relative fold to sham. One-way ANOVA was performed.

*P≤0.05; **P≤0.01; ****P≤0.0001.

### EXAMPLES

Brain endothelial loss of Pdzrn3 protects against induced BBB dysfunction and cognitive impairment whereas endothelial Pdzrn3 ectopic expression enhances brain damage in mice with chronic cerebral hypoperfusion.

Given that Pdzrn3 function is linked with reduced endothelial integrity, the inventors studied whether the cerebral phenotypes caused by chronic HP can be rescued by repression of or worsened by ectopic Pdzrn3 expression in central nervous system (CNS) endothelial cells (EC).

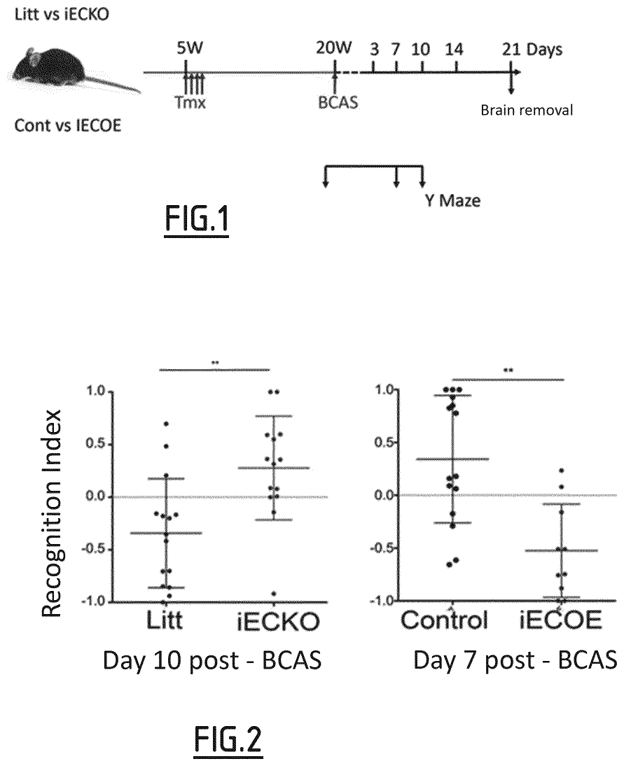

To recapitulate chronic HP insult, an experimental murine bilateral carotid artery stenosis (BCAS) model system was developed with ameroid constrictors implanted on both common carotid arteries (FIG. 1). Cortical surface Cerebral blood flow (CBF) was then recorded and a continued CBF decrease was observed at 21 days after surgery. Mice bearing a Pdzrn3 flox allele (Pdzrn3^{f/f}) were crossed with an EC-specific Pdgfb-CreER transgene to generate Pdzrn3^{f/f}; Pdgfb-iCreER (iECKO: inducible Endothelial Cells Knock Out) and Pdzrn3^{f/f} littermates (control). For this experiment recombination was induced with CreER and 4-hydroxytamoxifen (4HT) treatment 5 weeks after birth and animals were analyzed at 5 months (FIG. 1). Recombination efficiency in the brain was determined by analyzing Pdgfb-CreERT2; mTmG reporter mice showing that Pdgfb drives efficiently endothelial recombination in brain vessels. To avoid any effect of endothelial Pdzrn3 deletion on microvessel organization in CNS, the vasculature in brain volumes of iECKO mice vs control littermates was studied by light sheet microscopy.

Both Pdzrn3 iECKO and control littermate groups were then subjected to behavioral testing. To verify that the results from the cognitive tests were not confounded by differences in sensorimotor abilities between the animals, sensorimotor testing was first conducted. No statistical differences were observed between the groups in locomotor activity in terms of distance traveled, maximum speed, mean velocity. The two groups were subsequently tested, for spatial recognition memory in the Y-maze. Pdzrn3^{f/f} littermate group spent significantly less time in the novel arm compared to the iECKO group after BCAS at 10 days. Thus, Pdzrn3 deletion rescued the memory disturbance (FIG. 2, left side).

The effect of the endothelial Pdzrn3 deletion was studied on chronic HP-induced brain lesions and inflammation. Compared to controls, iECKO mice exhibited a significant decrease in the number of brain lesions (microinfarctions) as well as reduced neuronal loss in the CA1 hippocampal region known to play a crucial role in spatial recognition memory (FIG. 3, left side). To evaluate the effect of the Pdzrn3 deletion on brain inflammation, hippocampal GFAP expression was quantified, it is a marker of reactive astrocytes. Chronic HP induced an activation of astrocytes; loss of Pdzrn3 decreases GFAP positive labelling in the hippocampus (FIG. 4, left side).

Capillary leakage is a hallmark of altered BBB and found in clinic of AD or vascular dementia. To analyze BBB disruption under HP, hippocampal sections were triple stained for the endothelial and astrocyte markers, respectively Podocalyxin and GFAP, and with Fibrinogen to assess blood extravasation across leaky BBB. A rupture of both endothelial and astrocyte layers with extravasation of Fibrinogen was found in littermate while Pdzrn3-deficient mice showed reduced capillary rupture mostly in cortex and hippocampal regions. Furthermore, a higher accumulation of fibrinogen was found in mouse brain from littermates compared to iECKO mice under chronic HP (FIG. 5).

Maintenance of the BBB might requires functional interactions between endothelial cells, perivascular cell and astrocytes. Whole gliovascular unit transcripts were then analyzed by qRT-PCR in preparation of intact brain vessel fragments from the mouse brain parenchyma. Under 14 days of chronic HP, an increase of plasmalemma vesicle-associated protein (PVLAP), a marker for the high permeability endothelial state was observed, without significative modification of EC markers as Pecam, Cldn5 of Tjpn1. In the CNS, the canonical Wnt pathway is involved in EC to maintain BBB integrity via Wnt7a and 7b ligands. The expression of Wnt ligands was studied and that under chronic HP, a significant reduction of Wnt7b expression was reported but not of Wnt7a which may contribute to BBB stabilization.

Endothelial specific ectopic overexpression of Pdzrn3 (iECOE: inducible Endothelial Cells OverExpression) ability to accelerate vascular injury under hypoperfusion was assessed. In order to overexpress Pdzrn3 conditionally in a tissue specific manner, Pdgfb-CreER were crossed with ROSA:LNL:tTA transgenic mice (see Wang L, et al., Restricted expression of mutant SOD1 in spinal motor neurons and interneurons induces motor neuron pathology. Neurobiol Dis. 2008 March; 29(3):400-8) to generate Pdgfb-CreER/ROSA:LNL:tTA bigenic mice in which tTA was turned on specifically in EC. Then Pdgfb-CreER/ROSA:LNL:tTA mice were crossbred with TRE-Pdzrn3 mice (expressing PDZRN3-V5 and β galactosidase when tetO was activated by tTA). Both groups were obtained: Pdgfb-CreER/ROSA:LNL:tTA/tetO-Pdzrn3 triple-transgenic mice (Pdzrn3 OE), in which tTA activated tetO to express Pdzrn3 in EC and Pdgfb-CreER/ROSA:LNL:tTA (control group). For this experiment, recombination was induced with CreER and 4-hydroxytamoxifen (4HT) treatment 5 weeks after birth and animals were analyzed at 5 months. As expected, PDZRN3 and β galactosidase protein overexpression were detectable in cerebral vasculature. No modification of vessel organization in brain volumes in iECOE mice vs littermates was found by light sheet microscopy.

Data of the inventors showed that after 10 days of chronic HP, spatial recognition memory in iECOE group was even worse than those in control group (FIG. 2, right side). Chronic HP in iECOE resulted in global BBB damage with significant increase in brain microinfarct lesions, hippocampal neuronal loss (FIG. 3, right side), astrocytes activation (FIG. 4, right side) where IgG and fibrinogen extravasation into brain parenchyma in both cortical and hippocampal area were present (FIGS. 6 and 7).

Therefore, endothelial Pdzrn3 overexpression produces phenotypes opposite to those of endothelial Pdzrn3-deleted mice suggesting that PDZRN3-induced signaling tightly regulates barrier maintenance in the brain.

Loss of Pdzrn3 in endothelial cells attenuated Aβ deposit and reduces cognitive decline in a mouse model of AD.

As cerebral hypoperfusion is associated with the development of AD, it was studied whether endothelial PDZRN3 signaling contributes to worsening AD pathology. APPSwe/PSEN1dE9 mouse (APP/PS1) were crossed with transgenic Pdgfb-iCreER; Pdzrn3 to generate AD mouse cohorts deleted in endothelial cells for Pdzrn3 (APP/PS1; iECKO and their littermate control APP/PS1) and their respective non dementia (ND) littermate groups (ND-iECKO and ND-littermate controls).

A reduced cerebral blood flow was confirmed at 6 months which worsened at 12 months of age in both APP/PS1 and in APP/PS1; iECKO mouse groups. From 8 months, APP/PS1 mice were severely impaired in the spatial recognition memory paradigm whereas APP/PS1; iECKO littermates performed as well as ND-littermate control and ND-iECKO mice (FIG. 8). In APP/PS1 mouse model, amyloid plaques initially develop in the frontal cortex then spread to broad cortical and hippocampal regions. In accordance, with increasing age, APP/PS1 mice continuously accumulated more cortical and then hippocampal amyloid plaques. Strikingly, the plaque load (numbers and size) when Pdzrn3 is deleted at the age of 6 month was decreased in the cortex, and in both cortex and hippocampus at the age of 12 months in APP/PS1 mice (FIGS. 9 and 10). Abnormal activation of astrocytes and microglia has been related in human AD brains and in APP transgenic mouse model, which might increase the release of cytotoxic substances, thus causing further neuronal injury in the brain. An increase of GFAP immunolabeling at 6 months in both APP/PS1 littermates and APP/PS1; iECKO was observed; this activation was amplified at 12 months in APP/PS1 littermates but not in APP/PS1; iECKO cortical and hippocampal brain areas (see FIG. 11 for cortical area).

These findings demonstrate that the exacerbated Aβ deposits in APP/PS1 group leads to increase activation of astrocytes while deletion of Pdzrn3 in EC in reducing Aβ accumulation protects against astrogliosis.

Notably, in APP/PS1 littermate brains, large area of NeuN negative cortical cell nuclei correlated with an increase of IgG deposit at 12 months in brain sections were found; whereas NeuN negative large area in the cortex of APP/PS1; iECKO mice were not found (FIGS. 12-13). Increase extravasation of IgG in APP/PS1 brains (FIG. 13). was correlated with a rupture of BBB as reported in cortical sections from APP/PS1 triple stained for the endothelial and astrocyte markers, respectively Podocalyxin and GFAP, and with Fibrinogen to assess blood extravasation across leaky BBB while APP/PS1; Pdzrn3-deficient mice showed reduced capillary rupture.

These results suggest that deletion of Pdzrn3 in reducing extravasation of blood proteins in the cerebral parenchyma protects against neuronal death.

Pdzrn3 Induced Pathway Regulates Barrier-Specific Claudin5 and Wnt7b Gene Expression in Cerebral Vasculature

It was proposed that the level of endothelial junctional proteins decrease or relocalize in BBB dysfunction associated with CNS diseases. PDZRN3 was implicated in junctional protein polarized localization and maintain in brain EC. As accumulating evidence point to a correlation between BBB dysfunction and alteration of endothelial tight junction (TJ) complexes, whether endothelial deletion of Pdzrn3 regulates barrier specific protein expression in AD context was studied.

In intact brain vessel fragment preparation, characterization of brain vasculature in APP/PS1 mice revealed a reduced expression of Wnt7b as previously found under chronic hypoperfusion in the BCAS model; however endothelial deletion of Pdzrn3 can rescue the effect of APP/PS1 and induced Wnt7b in gliovascular units. As altered expression of Claudin 5 has been linked to a reduced Wnt canonical signaling in the CNS, the expression of endothelial cell junctional markers were studied. Endothelial deletion of Pdzrn3 induced a strong enrichment of the expression of Cldn5 both at the mRNA (FIG. 14) and protein levels in APP/PS1 mice compared to that in APP/PS1 littermate controls; in contrast, the other BBB markers such as ZO1 and occludin were not significantly modified. Confocal immune microscopy was used to characterize patterns of Claudin-5 expression at EC junctions on isolated SMA negative-capillaries. In line with our earlier finding, junctional Claudin 5 staining intensity decreased in APP/PS1 compared to that in ND-age matched control mice. Additionally, Claudin 5 was more abundant into EC-EC contact sites in brain microvessels from APP/PS1; iECKO mutant vs APP/PS1littermate control mice. The distribution of VE cadherin appeared not modified in the vessel preparations. VE cadherin was detected at equivalent levels in capillaries both from APP/PS1 vs ND-littermates and from APP/PS1-iECKO vs APP/PS1.

These data suggest that down regulation of PDZRN3 signaling leads to increase TJ strands with a reduction of vascular permeability.

These finding lends strong support to a model in which AD pathology compromises BBB Wnt pathway signalling and in which under AD context, loss of Pdzrn3 in endothelial cells maintains BBB state in a more “healthy” state.

1. A method for preventing and/or treating a disease linked with cerebral hypoperfusion.

2. The method according to claim 1, wherein the disease is selected from vascular dementia, Parkinson's disease, Multiple sclerosis or Alzheimer's disease.

3. The method according to claim 1, wherein said inhibitor is an inhibitor of Pdzrn3 expression.

4. The method according to claim 3, wherein said inhibitor is chosen from small interfering (siRNA), small hairpin RNA (shRNA), micro RNA (miRNA), antisense oligonucleotides and aptamers.

5. The method according to claim 1, wherein said inhibitor inhibits PDZRN3 biological activity or inhibits PDZRN3 interaction with its targets.

6. The method according to claim 5, wherein said inhibitor is chosen from chemical molecules, peptides, proteins, aptamers, antibodies and antibody fragments.

7. The method according to claim 1, wherein said inhibitor reduces brain inflammation.

8. The method according to claim 1, wherein said inhibitor is fused or linked to a tag allowing the targeting of cerebral endothelial cells.

9. The method according to claim 1, wherein said inhibitor is formulated in a pharmaceutical composition.

10. The method according to claim 1, wherein said inhibitor is administered by oral route, intranasal route, intraocular route, parenteral route, or by intramuscular, subcutaneous, intravenous, intraperitoneal or local injections.

11-12. (canceled)

13. An in vitro screening method for the identification of a candidate compound suitable for preventing and/or treating disease linked with cerebral hypoperfusion, said method comprising:
a. culturing endothelial cells, in the presence and in the absence of a candidate compound;
b. measuring the level of expression of Pdznr3 or the level of PDZRN3 biological activity in endothelial cells cultured in the presence and in the absence of the candidate compound;
c. comparing the level of expression of Pdznr3 in endothelial cells in the presence the candidate compound, with the level of expression of Pdznr3 endothelial cells in the absence of the candidate compound;
or comparing the level of PDZRN3 biological activity in endothelial cells in the presence the candidate compound, with the level of PDZRN3 biological activity in endothelial cells in the absence of the candidate compound; and
d. identifying the candidate compound as suitable for preventing and/or treating disease linked with cerebral hypoperfusion if the level of expression of Pdznr3 in endothelial cells in the presence the candidate compound is decreased compared with the level of expression of Pdznr3 in endothelial cells in the absence of the candidate;
or identifying the candidate compound as suitable for preventing and/or treating disease linked with cerebral hypoperfusion if the level of PDZRN3 biological activity in endothelial cells in the presence the candidate compound is decreased compared with the level PDZRN3 biological activity in endothelial cells in the absence of the candidate.

14. A pharmaceutical composition comprising a PDZRN3 inhibitor and a pharmaceutically acceptable carrier.
